(19) United States
(12) Patent Application Publication
(10) Pub. No.: US 2023/0347107 A1
(43) Pub. Date: Nov. 2, 2023

(54) BALLOON-ASSISTED NEUROVASCULAR THROMBECTOMY DUAL ASPIRATION AND DISTAL ACCESS CATHETER SYSTEM

(71) Applicant: The Research Foundation for the State University of New York, Albany, NY (US)

(72) Inventor: Hesham Masoud, Syracuse, NY (US)

(21) Appl. No.: 18/140,240

(22) Filed: Apr. 27, 2023

Related U.S. Application Data
(60) Provisional application No. 63/336,465, filed on Apr. 29, 2022.

(51) Int. Cl. A61M 25/00; A61M 25/10

(52) U.S. Cl. CPC A61M 25/0026; A61M 25/0068; A61M 25/10185; A61M 2025/1052

(57) ABSTRACT

A catheter system that allows balloon occlusion for flow-arrest assisted aspiration in the intracranial circulation with direct aspiration. The system has an inner coaxial floppy intra-cranial aspiration catheter that allows for direct thrombectomy of proximal and distal vessel blockages in a single device, through direct or remote aspiration with enhanced flow arrest of the intracranial circulation via balloon inflation. Rapid deflation of the balloon is achieved with withdrawal of the inner aspiration on thrombus retrieval, for seamless reperfusion of the vessel, and the system can provide continuous aspiration via an outer aspiration catheter. Automatic deflation of the balloon occurs with retrieval of the inner co-axial floppy intra-cranial aspiration catheter under continuous aspiration to guard against prolonged balloon time inflation and risk of vessel injury during the revascularization procedure.

### CROSS-REFERENCE TO RELATED APPLICATION

This application claims the benefit of U.S. Provisional Patent Application No. 63/336,465, filed on Apr. 29, 2022, the entirety of which is hereby incorporated herein by this reference.

### BACKGROUND OF THE INVENTION

### 1. Field of the Invention

The present invention generally relates to catheter systems. More particularly, the present invention relates to a balloon assisted thrombectomy dual aspiration and distal access catheter system for neurovascular thrombectomy and intracranial emboli protection.

### 2. Description of the Related Art

In general, Current technology employs use of stent retriever devices that require use of micro catheter and micro wire for deployment of the device which is then unsheathed and retrieved through the intracranial circulation, this can lead to increased time to target vessel prolonging time of therapy and retrieval of the stent device can risk vessel injury or re-occlusion of the vessel from scraping of unstable plaque on device retrieval. Aspiration catheters provide an alternative to use of stent retrievers, with device sizes depending on size of vessel blockage and allow for direct aspiration of the blockage. Use of an adjunctive proximal balloon guide catheter, which allows for flow arrest in the extra cranial vessels, is associated with enhanced reopening of blocked vessels when used with the intracranial devices mentioned above. However, these balloon guide catheters require separate time for preparation, and are relatively inflexible for advancement in tortuous anatomy and not designed for use beyond extracranial vessels.

Intracranial angioplasty and stenting procedures are associated with distal embolic strokes due to disruption and fragmentation of plaque debris that can be carried in the more distal brain vessels causing stroke, currently there exists no form of intracranial protection against these emboli. Innovation in the field of these devices is needed as the time to reopen the vessel is known to be associated with outcomes for improved stroke patient functional independence in these disabling strokes due to intracranial blockages. It is thus to address this problem that the present invention is primarily directed.

### BRIEF SUMMARY OF THE INVENTION

Briefly described, the device presented herein is intended for use in endovascular treatment of intra-cranial vessel occlusions. The device is designed for rapid atraumatic advancement in intracranial vasculature, and aspiration with balloon flow arrest for removal of intravascular blockages. The device allows for transient flow arrest of an intracranial vessel and use of the device as distal emboli protection for intra-cranial vessel angioplasty and stenting procedures. The device may also be used in other neuro endovascular procedures such as balloon test occlusions to assess vasculature viability before vessel sacrifice.

The present disclosure provides a stroke treatment device that includes balloon occlusion for flow-arrest assisted aspiration in the intracranial circulation with direct aspiration, an inner coaxial floppy intra-cranial aspiration catheter allowing for direct thrombectomy of proximal and distal vessel blockages in a single device, through direct or remote aspiration with enhanced flow arrest of the intracranial circulation via balloon inflation. Rapid deflation is achieved with withdrawal of the inner aspiration on thrombus retrieval, for seamless reperfusion of the vessel, and added option of continuous aspiration via the outer aspiration catheter. Automatic deflation of the balloon occurs with retrieval of the inner co-axial floppy intra-cranial aspiration catheter under continuous aspiration, this guards against prolonged balloon time inflation and risk of vessel injury during the revascularization procedure. The inner aspiration catheters distal floppy segment, is designed for a dramatic advancement in intracranial circulation without need for coaxial micro catheter or wire, thus allowing for more rapid advancement and retrieval of blockages. Further, the distal balloon aspiration catheter can be employed in intracranial angioplasty and stenting procedures, as a more proximal embolic protection device through its flow arrest function, the inner aspiration catheter may serve as an intermediate catheter for deployment of stiffer intracranial stents systems, balloon expanding or self-expanding endovascular stents.

A first aspect of the disclosure provides a catheter system, including: an inner aspiration catheter having a first lumen; and a balloon aspiration catheter including: a balloon having a second lumen, a body, a distal tip, and a proximal end, a primary aspiration channel connected to the first lumen of the balloon, a plurality of balloon inlets disposed along the body of the balloon, and a secondary inflation channel connected to the body of the balloon.

A second aspect of the disclosure provides a catheter system, including: an inner aspiration catheter having a first lumen, and a balloon aspiration catheter including: a balloon having a second lumen, a body, a distal tip, and a proximal end, a primary aspiration channel connected to the first lumen of the balloon, a plurality of balloon inlets disposed along the body of the balloon, and a secondary inflation channel connected to the body of the balloon. The balloon of the balloon aspiration catheter is configured such that the balloon inflates when the inner aspiration catheter is advanced beyond the balloon inlets and an operator injects a solution into the secondary inflation channel of the balloon.

Balloon aspiration in the intracranial circulation results in a stronger vacuum and subsequent suction effect for retrieval of more distal emboli (e.g. emboli in two branches sharing the same trunk) through a remote aspiration technique. The coaxial inner floppy aspiration catheter allows for retrieval of more distal occlusions with direct aspiration further augmented by balloon flow arrest from the outer catheter. Withdrawal of the inner catheter proximal to the balloon outer marker results in rapid deflation of the balloon for restoration of flow and continued aspiration via the outer catheter. Another advantage is the speed of device deployment and ease of use, with design for rapid advancement in cerebrovascular anatomy without use of coaxial support due to atraumatic flexible distal segment of the inner catheter. The device is compatible with other methods of thrombectomy including stent retriever use, and deployment of intracranial balloon angioplasty and stenting devices. The device can also be used as a distal emboli protection device as the balloon inflation causes flow arrest and suction effect can be applied via the aspiration catheter for flow reversal to prevent distal embolization of fragments of intracranial atherosclerotic plaque.

The present invention is therefore advantageous as it allows rapid treatment of treatment of intra-cranial vessel occlusions. Furthermore, the present invention is industrially applicable in that it is a manufacturable medical device that provides medical treatment. Other advantages and features of the present invention will be apparent to one of skill in art.

### BRIEF DESCRIPTION OF THE DRAWINGS

FIG. 1A is an illustration of trans femoral or trans-radial access for percutaneous treatment of an intracranial clot.

FIG. 1B is an illustration an example of the use of the balloon aspiration device for flow arrest-assisted aspiration thrombectomy

FIG. 1C is an illustration of the pressure differential in the intracranial clot of FIG. 1B.

FIG. 2A shows the inner aspiration catheter parts, in sagittal plane and in connection with the outer balloon aspiration catheter.

FIG. 2B illustrates the proximal inner aspiration catheter hub and parts of the catheter system.

FIG. 2C illustrates a sagittal plane transection of the device showing the inner aspiration catheter structure and inner lumen.

FIG. 2D illustrates a cross-section of the inner aspiration catheter structure and the inner lining.

FIG. 2E illustrates a cross-section of the inner aspiration catheter structure with and the outer lining.

FIG. 3A illustrates the outer balloon aspiration catheter in sagittal plane.

FIG. 3B illustrates the coaxial fitting of the inner aspiration catheter for balloon inflation.

FIG. 3C illustrates the balloon aspiration catheter proximal parts and in conjunction with inner aspiration catheter.

FIG. 3D illustrates the device in several cross-sectional planes.

FIG. 3E illustrates the coaxial fitting of the inner aspiration catheter for balloon inflation.

FIG. 4A shows the assembled balloon aspiration catheter system with magnified illustrations showing the inflation balloon mechanism.

FIG. 4B illustrates the deflation mechanism of the outer aspiration catheter balloon using the coaxial inner aspiration catheter.

FIG. 4C illustrates the assembled balloon aspiration thrombectomy catheter system showing the inner and outer aspiration balloon catheters and separate connection hookups for the outer balloon inflation syringe, and primary lumen inner and outer catheter aspiration.

FIG. 5A shows an example of the device use for thrombectomy and its mechanism of balloon flow arrest assisted thrombectomy using the co-axial inner aspiration catheter and deflation of the balloon by withdrawal of the aspiration catheter.

FIG. 5B is a further illustration of 5A showing a magnified view to illustrate remote and direct aspiration with balloon assisted flow arrest and spontaneous balloon deflation with inner aspiration catheter withdrawal and continued aspiration via the outer balloon aspiration catheter for retrieval of thrombus and thrombus debris.

FIG. 5C is a further illustration of the procedure of FIGS. 5A-5B.

FIG. 5D further illustration of the procedure in FIGS. 5A-5C.

FIG. 5E further illustration of the procedure in FIGS. 5A-5D.

FIG. 5F further illustration of the procedure in FIGS. 5A-5E.

FIG. 5G further illustration of the procedure in FIGS. 5A-5F.

FIG. 5H further illustration of the procedure in FIGS. 5A-5G.

FIG. 6A illustrates the device being an intracranial emboli protection device during treatment of intracranial atherosclerotic disease with the device's balloon inflation for flow arrest to minimize antegrade flow during navigation of secondary wires and devices to treat intracranial atherosclerotic plaque, such as balloon angioplasty.

FIG. 6B illustrates the inner and outer catheters of the device utilized in the procedure of FIG. 6A.

### DETAILED DESCRIPTION OF THE INVENTION

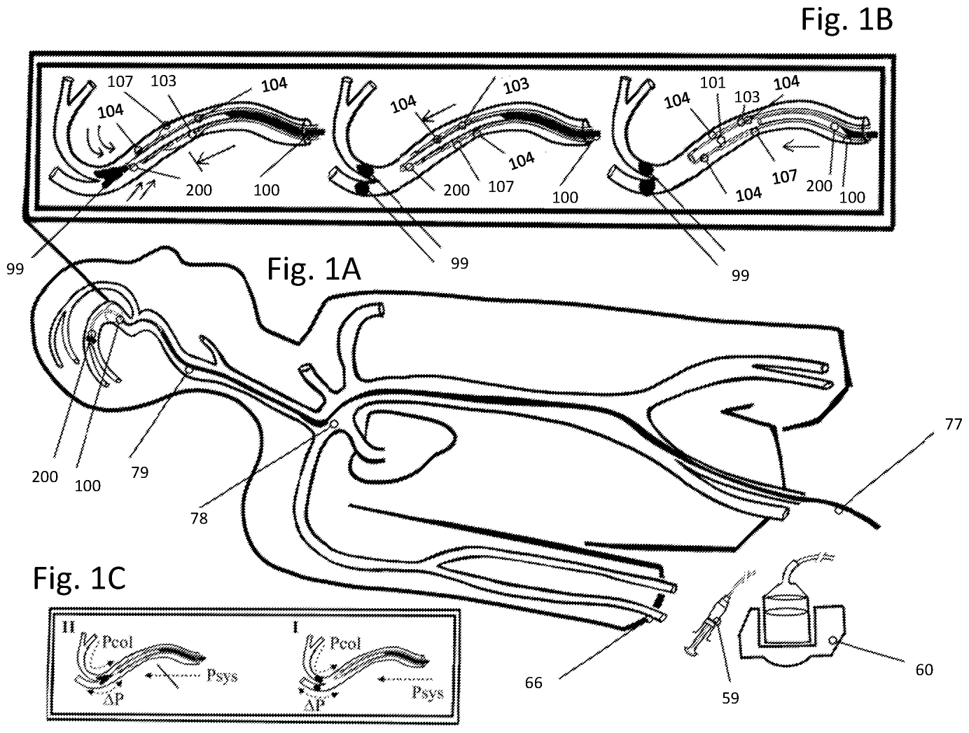

With reference to the figures in which like numerals represent like elements throughout the several views, FIG. 1A is an illustration of trans femoral or trans-radial access for percutaneous treatment of an intracranial clot in a patient. FIG. 1B is an illustration of an example of the use of the balloon aspiration device 100 for flow arrest-assisted aspiration thrombectomy. FIG. 1C is an illustration of the pressure differential in the intracranial clot of FIG. 1B.

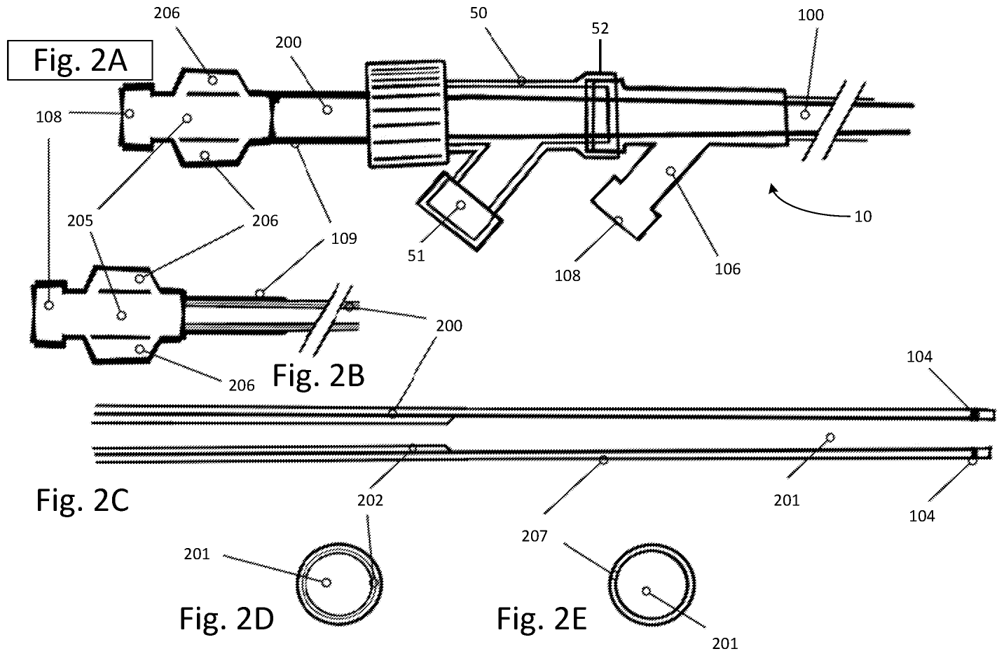

FIGS. 2A-E further illustrate the design of the present catheter system 10. FIG. 2A shows the inner aspiration catheter parts, in sagittal plane and in connection with the outer balloon aspiration catheter. FIG. 2B illustrates the proximal inner aspiration catheter hub 205 and parts of the catheter system 10. FIG. 2C illustrates a sagittal plane transection of the device showing the inner aspiration catheter structure and inner lumen 201. FIG. 2D illustrates a cross-section of the inner aspiration catheter structure and the inner lining 202. FIG. 2E illustrates a cross-section of the inner aspiration catheter structure with and the outer lining 207.

The catheter system 10 is employed in the endovascular treatment of ischemic cerebrovascular disease, namely intracranial atherosclerotic disease and large vessel intracranial occlusions. The catheter system 10 is prepared and assembled ex vivo prior to use in the patient's intracranial circulation, the inner catheter 201 and outer catheter 101 lumens are flushed with saline filled syringes attached via their respective catheter hubs, the outer balloon aspiration catheter balloon port hub 52 is also manually flushed, but with a saline contrast mix to prime the outer balloon aspiration catheter secondary lumen 110 which communicates with the balloon 107. A standard rotating hemostatic valve 50 is affixed to the outer balloon aspiration catheter hub 105 utilizing standard Luer fitting 108, and the rotating hemostatic valve side port is attached to a three-way stopcock 53 for continuous antegrade saline flush line drip and aspiration via aspiration syringe 61 or pump machine 60. The outer balloon aspiration catheter hub 105 balloon side port 106 is attached to a one-way stopcock 52 and 1 mL syringe 59 filled with contrast saline mix.

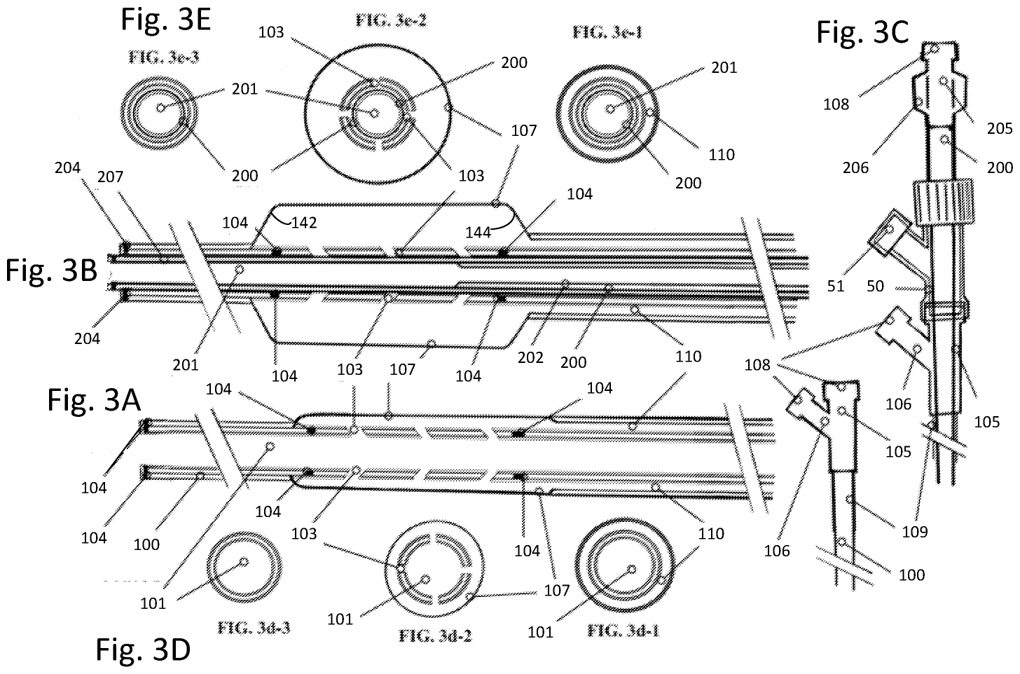

FIG. 3A illustrates the outer balloon aspiration catheter 100 in sagittal plane. FIG. 3B illustrates the coaxial fitting of the inner aspiration catheter 200 for balloon 107 inflation. FIG. 3C illustrates the balloon aspiration catheter 100 proximal parts and in conjunction with inner aspiration catheter 200. FIG. 3D illustrates the device in several cross-sectional planes across FIG. 3A, with FIGS. 3d-1 to 3d-3 representationally adjacent to the cross-sectional plane location of FIG. 3A. FIG. 3E illustrates the coaxial fitting of the inner aspiration catheter for balloon inflation, with FIGS. 3e-1 to 3e-3 representationally adjacent to FIG. 3B at cross-sectional locations, illustrating coaxial fitting of the inner aspiration catheter 200 for balloon 107 inflation.

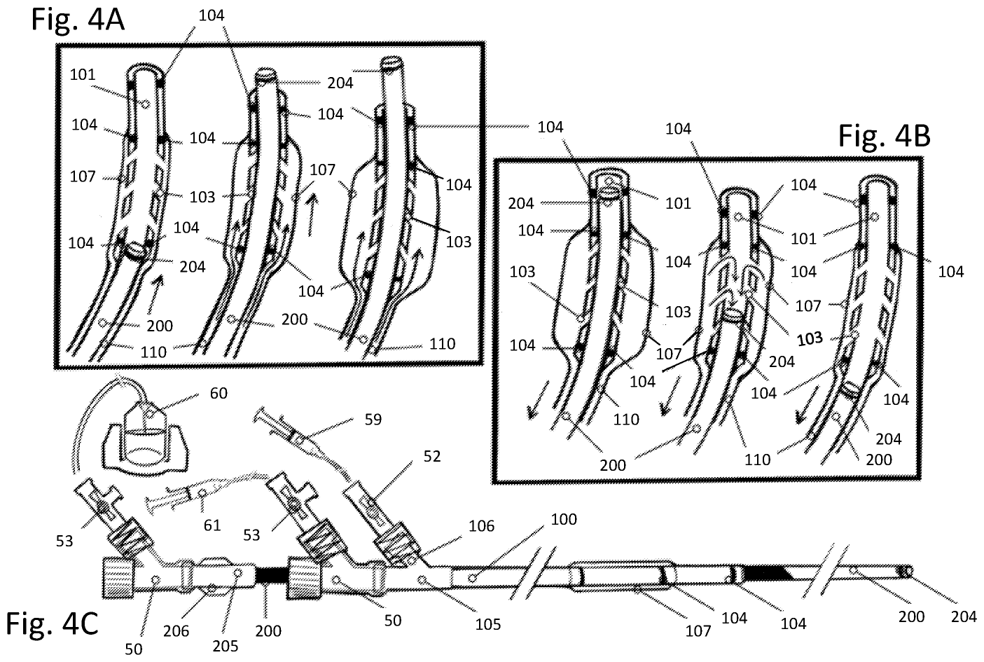

The inner aspiration catheter 200 fits coaxially into, and extends beyond, the outer balloon aspiration catheter 100 to occlude the outer catheter inner lumen 101 balloon ports/inlets 103, as illustrated in FIG. 3A-3E, and allow for inflation and deflation of the balloon 107 with contrast saline mix via the secondary lumen 110 connected to the balloon aspiration catheter hub 105 side port 106 administered with 1 way stop cock 52 and syringe 59. Rapid deflation of the balloon 107 is also possible with withdrawal of the inner aspiration catheter 200 proximal to the balloon side ports/inlets 103 resulting in release of trapped contrast saline mix into the balloon aspiration catheter 100 primary lumen 101 under aspiration via the balloon aspiration catheter hub 105 attached to a rotating hemostatic valve 50 with side port 51 attachment to a syringe 59 or machine pump 60 for aspiration of the balloon aspiration catheter 100 primary lumen 101, as illustrated in FIGS. 4A-4C.

FIG. 4A shows the assembled balloon aspiration catheter system with magnified illustrations showing the inflation balloon 107 mechanism. FIG. 4B illustrates the deflation mechanism of the outer aspiration catheter balloon 107 using the coaxial inner aspiration catheter 200. FIG. 4C illustrates the assembled balloon aspiration thrombectomy catheter system showing the inner catheter 200 and outer aspiration balloon catheter 100 and separate connection hookups for the outer balloon inflation syringe 59, and primary lumen 101 inner and outer catheter aspiration.

The inner aspiration catheter 200 is introduced via the rotating hemostatic valve 50 into the outer balloon aspiration catheter 100 primary lumen 101, and advanced beyond the catheter tip. The inner aspiration catheter 200 is also connected to a rotating hemostatic valve 50. The rotating hemostatic valve side port is also attached to a three-way stopcock 53 for the purposes of continuous saline flush of the inner aspiration catheter primary lumen and for aspiration via a syringe 61 or machine pump 60.

In one embodiment, the assembled catheter system is introduced into a 6 french guide catheter 79 (FIG. 1A) that has been navigated via a transfemoral 77 or alternate trans-radial 66 route across the aortic arch 78, above the heart and into the great cervical vessels to access the anterior or posterior circulation via the respective carotid or vertebral arteries.

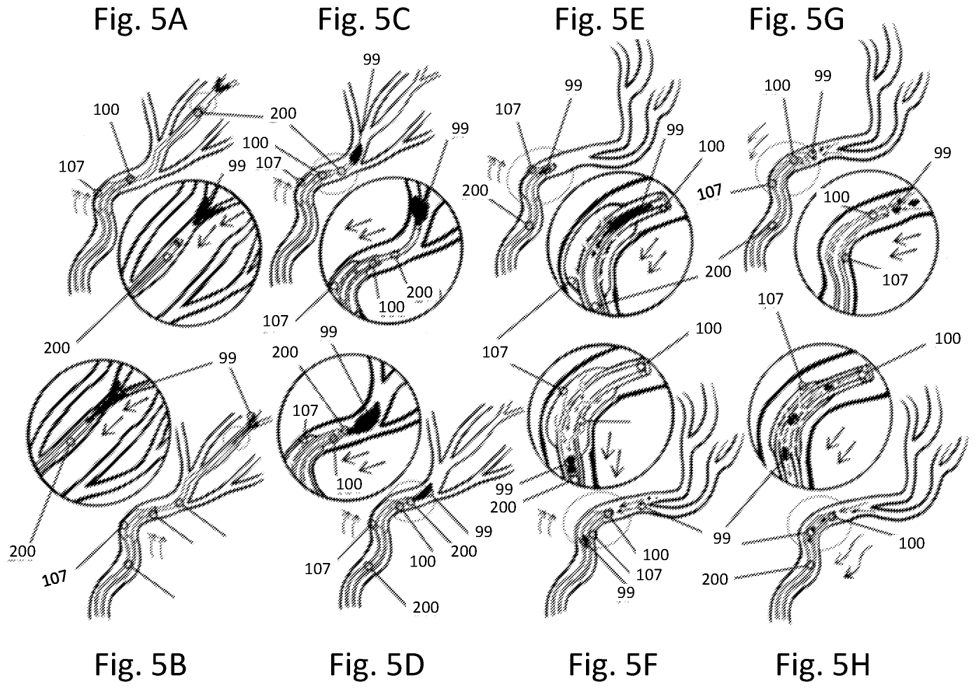

The catheter system can be advanced coaxially without the support of a wire or micro catheter in uncomplicated anatomy due to the compliant distal tip of the inner aspiration catheter which allows for atraumatic navigation with advancement of the catheter. The dual catheter system is advanced in tandem with the inner aspiration catheter 200 advanced beyond the outer balloon aspiration catheter 100, with atraumatic navigation facilitated by the compliant distal tip, allowing for navigation past the para-ophthalmic internal carotid artery for direct aspiration of intracranial occlusions spanning from the middle cerebral artery trunk to the distal insular branches, as illustrated in FIGS. 5A-5G.

FIG. 5A shows an example of the system's 10 use for thrombectomy and its mechanism of balloon flow arrest assisted thrombectomy using the co-axial inner aspiration catheter 200 and deflation of the balloon 107 by withdrawal of the aspiration catheter. FIG. 5B is a further illustration of FIG. 5A showing a magnified view to illustrate remote and direct aspiration with balloon 107 assisted flow arrest and spontaneous balloon deflation with inner aspiration catheter 200 withdrawal and continued aspiration via the outer balloon aspiration catheter 100 for retrieval of thrombus and thrombus debris. FIG. 5C-5G are further illustrations of the aspiration procedure.

The outer balloon aspiration catheter 100 is advanced over the inner aspiration catheter 200, and inflated using contrast saline mix via the outer balloon aspiration catheter secondary lumen 110 which is connected to the balloon aspiration catheter hub side port 106, and administered via a 1 mL syringe 59 attached with a one way stopcock 52.

The outer balloon aspiration catheter 100 may be utilized alone as a primary aspiration catheter, with its larger bore for proximal occlusion direct aspiration without the functionality of balloon inflation given its use without coaxial inner aspiration catheter. The inner aspiration catheter distal tip is without an inner lining 202 at its distal most segment as such allowing for direct advancement without need of support of coaxial micro catheter allowing for simpler rapid delivery to target occlusions. The inner aspiration catheter 200 length and diameter are sized to access the distal insular segments of the middle cerebral artery for thrombectomy of medium sized vessel occlusions.

Inflation of the balloon 107 in the intracranial carotid artery and proximal middle cerebral artery provides antegrade flow arrest to enhance local and remote aspiration thrombectomy by creating an atraumatic vacuum seal in parent vessel intracranial circulation and a stronger aspiration force to retrieve thrombus with a direct or remote thrombectomy aspiration thrombectomy approach via the inner aspiration catheter 100 placed proximal to distal emboli that share parent vessel bifurcation, as illustrated in FIGS. 5A-5H.

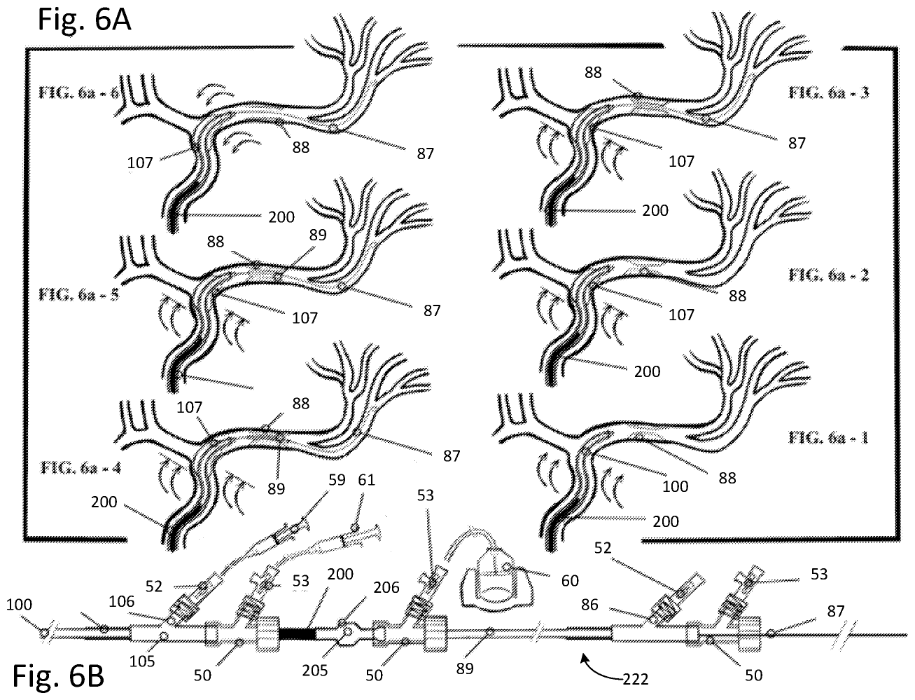

The dual catheter system 10 can also be used to assist in the endovascular treatment of intracranial atherosclerotic disease due to its dual function as a distal access catheter (via the inner aspiration catheter 200 inner lumen 201 that is compatible with standard outer diameter intracranial balloon 89 and stent devices, the inner aspiration catheter 200 provides support for delivery of stiff devices, and the outer balloon aspiration catheter 100 balloon inlets 103 inflation provides embolic protection with antegrade flow arrest, used to prevent debris from embolizing during the critical stages of traversing an intracranial narrowed lesion with a microwire 87, balloon angioplasty using balloon catheter hub side port 86 with 1 way stop cock 53 and 1 ml syringe, for balloon angioplasty with or without intracranial stenting interventions of unstable intracranial plaque and vessel stenosis, after the chosen treatment aspiration is performed via the inner aspiration catheter (200), and before outer aspiration catheter balloon deflation, as illustrated in FIG. 6a1-6. Thus, providing protection against embolization with flow arrest and prevention of embolic stroke with aspiration prior to restoration of blood flow.

FIG. 6A illustrates the device 222 being an intracranial emboli protection device during treatment of intracranial atherosclerotic disease with the device's balloon 107 inflation for flow arrest to minimize antegrade flow during navigation of secondary wires (microwire 87) and devices to treat intracranial atherosclerotic plaque, such as balloon angioplasty. FIG. 6B illustrates the inner catheter 100 and outer catheter 200 of the device 222 utilized in the procedure of FIG. 6A.

Furthermore, subfigures FIGS. 6a-1 to 6a-6 illustrate the device being an intracranial emboli protection device during treatment of intracranial atherosclerotic disease with the device's balloon inflation for flow arrest to minimize antegrade flow, during navigation of secondary wires and devices to treat intracranial atherosclerotic plaque, such as balloon angioplasty, and further shows the device's 222 use as an embolic protection device for aspiration of debris caused by treatment of unstable intracranial plaque. Subfigures FIGS. 6a-1 to 6a-6, in conjunction with device 222, illustrate a co-axial balloon angioplasty microcatheter and microwire illustrated to show the device's use as a secondary embolic protection device in the treatment of intracranial plaque.

The balloon 107 rapidly deflates with withdrawal of the inner aspiration catheter 200 proximal to the balloon side ports/inlets 103, as previously trapped contrast saline mix is allowed to escape into the primary lumen 101, the balloon aspiration catheter primary lumen 101 is also under aspiration using syringe 61 or machine pump 60, performed via side port on the rotating hemostatic valve 50 attached to its hub 105, as such there is continuous protection of emboli during the withdrawal of the inner aspiration catheter 200 and flow restoration without prolonged balloon occlusion, as illustrated in FIGS. 4A-4B and FIGS. 5F-5H.

To build, produce, and/or manufacture the device 222, the inner aspiration catheter 200 may include a single lumen flexible catheter with a braided, helical or coil design using low friction lubricious tubing, the catheter distal segment (10 to 15 cm) is soft due to lack of inner PTFE lining in the distal segment to achieve increased flexibility and softness. The effective length of the catheter is approximately 150 cm with the distal length being approximately 15 cm and a proximal length of approximately 135 cm. The inner diameter of the catheter is approximately 0.040 to 0.055 inches with an outer diameter of approximately 0.055 to 0.070 inches. The proximal hub side wings 206 and standard Luer fitting made of nylon or similar materials. The catheter size may be any French size but ideally 5 French and may also be compatible with standard micro catheter sizes or similar. Radiopaque markers are positioned approximately 1 to 2 mm from the distal catheter tip. The catheter may have a straight tip that is steam shapable.

The outer aspiration balloon catheter 100 may include a single lumen flexible catheter, utilizing low friction tubing and braid, or helical or coil design. The balloon is compliant made of polyurethane, silicone, or similar and located 3 to 5 mm from the distal catheter tip. The balloon has two radiopaque markers delineating the length of the balloon and there is a distal radiopaque marker of the catheter tip. The compliant balloon size diameter are approximately 2 to 3 mm and have a length of approximately 6 to 9 mm. The proximal hub is made of a material such as nylon, includes standard-size wings, and a Luer fitting or similar. The effective catheter length is approximately 135 cm with a distal length of approximately 15 cm, and a proximal length of approximately 120 cm. The inner diameter of the catheter is approximately 0.071-0.74 inches and an outer diameter of approximately 0.084 inches. The catheter size may be 6 French and include a straight tip.

The catheter outer layer could be manufactured using a combination of Polyblend and Pellethane, Pebax (Polyether block amide, thermoplastic elastomer) and Grilamid (high-performance polyamide), or Pebax with Nylon, or similar materials and/or combinations. The inner layer may include stainless steel braid, helical, coil, PTFE (Polytetrafluoroethylene) and polyolefin elastomer, stainless steel with Nitinol wire and polymer fiber braid and coil, or similar materials. The catheter portions may include a hydrophilic coating (Hydak®). Radiopaque markers may comprise of Platinum, Iridium, or similar. The hub portion may include Nylon or similar materials. The catheters stress relief jacket may be made of polyurethane or similar materials. The catheter introducer may be made from Pebax or similar materials.

In embodiments, the catheter system 10 can includes a first hemostatic valve 50 having a first hemostatic valve side port and a proximal end, an inner aspiration catheter hub 205 connected to the proximal end of the first hemostatic valve 50, an inner aspiration catheter shaft 200 connected to the inner aspiration catheter hub 205, the inner aspiration catheter shaft having an interior thereof, and including an inner lumen 201 formed within the interior of the inner aspiration catheter shaft, an inner lining 202 fitted within at least a portion of the inner lumen 201, the inner lining 202 having an outer lining 207, and a stress relief jacket 109 fitted over the outer lining, an outer balloon aspiration catheter hub 105, including a second hemostatic valve 50 having a second hemostatic valve side port 51, and a third hemostatic valve having a third hemostatic valve side port 106. There is an outer balloon aspiration catheter shaft 100 having an interior thereof, and include a primary lumen 101 formed within the interior of the outer balloon aspiration catheter shaft and connected to the second hemostatic valve 50, and the primary lumen further having an exterior surface. A secondary lumen 110 is fitted over the primary lumen and connected to the third hemostatic valve, and the secondary lumen has a secondary lumen side port 106 and an interior thereof, and an expandable balloon 107 formed within the interior of the secondary lumen, the balloon having an interior thereof and a plurality of balloon side ports/inlets 103 disposed along the interior of the balloon and a portion of the exterior of the primary lumen 101, and wherein, the plurality of balloon side ports connect the primary lumen 101 to the secondary lumen 201 such that the plurality of balloon side ports expand the expandable balloon from a pressurized fluid and contract the expandable balloon upon removal of the inner aspiration catheter shaft 201.

In embodiments, the inner aspiration catheter shaft 200 is hydrophilic and the inner aspiration catheter shaft 200 has a lubricious outer coating. In other embodiments, a plurality of radiopaque markers 104 are disposed throughout the inner lumen, the primary lumen, and the secondary lumen. In embodiments, the first hemostatic valve, the second hemostatic valve, and the third hemostatic valve each include a Luer fitting. In further embodiments, the first hemostatic valve side port, the second hemostatic valve side port, and the third hemostatic valve side port each include at least one of a one-way stop cock 52 and a three-way stop cock 53. A machine pump 60 can be connected to the at least one of a one-way stop cock 52 and a three-way stop cock 53. In embodiments, a syringe 59 is connected to the at least one of a one-way stop cock 52 and a three-way stop cock 53.

In embodiments, the syringe is an aspiration syringe and the inner aspiration catheter hub 205 further comprises at least two hub side wings 206. In embodiments, the inner aspiration catheter hub further comprises a standard Luer fitting 108 and the outer lining is supported with one or more of the group comprised of embedded coils and wire braid. The catheter system 10 can further include an intracranial balloon 89 fitted within the inner lumen of the inner aspiration catheter shaft; a fourth hemostatic valve 86 having a fourth hemostatic valve side port and connected to the inner aspiration catheter shaft, a fifth hemostatic valve 50 having a fifth hemostatic valve side port and connected to the fourth hemostatic valve, and a microwire 87 fitted within the inner lumen and the intracranial balloon.

In other embodiments, the first hemostatic valve, the second hemostatic valve, and the third hemostatic valve each include a Luer fitting, and the microwire 87 includes a curved distal tip 88. The fourth hemostatic valve side port and the fifth hemostatic valve side port can each include at least one of a one-way stop cock 52 and a three-way stop cock 53. Further, the machine pump 60 can be connected to the at least one of a one-way stop cock 52 and a three-way stop cock 53. In embodiments, a syringe 59 is connected to the at least one of a one-way stop cock 52 and a three-way stop cock 53.

In further embodiments, the catheter system 10 includes a first hemostatic valve 50 having a first hemostatic side port and a proximal end, an inner aspiration catheter hub 205 connected to the proximal end of the first hemostatic valve 50, an inner aspiration catheter shaft 200 connected to the inner aspiration catheter hub 205, the inner aspiration catheter shaft having an interior thereof. There is an outer balloon aspiration catheter hub 105 and an outer balloon aspiration catheter shaft 100, including a primary lumen 101 formed within the interior of the outer balloon aspiration catheter shaft and connected to the second hemostatic valve 50, and the primarily lumen having an exterior surface. A secondary lumen 110 is fitted over the primary lumen and connected to the third hemostatic valve, wherein the secondary lumen has a secondary lumen side port 106 and an interior thereof, with an expandable balloon 107 formed within the interior of the secondary lumen, the balloon having an interior thereof and a plurality of balloon side ports/inlets 103 disposed along the interior of the balloon and a portion of the exterior of the primary lumen 101.

In such embodiment, the plurality of balloon side ports connect the primary lumen 101 to the secondary lumen 201 such that the plurality of balloon side ports expand the expandable balloon from a pressurized fluid and contract the expandable balloon upon removal of the inner aspiration catheter shaft 201. In some embodiments, the inner aspiration catheter shaft has an inner lumen 201 formed within the interior of the inner aspiration catheter shaft, with an inner lining 202 fitted within at least a portion of the inner lumen 201, the inner lining having an outer lining 207 over the inner lining 202, and a stress relief jacket 109 fitted over the outer lining.

In other embodiments, the outer balloon aspiration catheter hub further has a second hemostatic valve 50 having a second hemostatic valve side port 51, and a third hemostatic valve having a third hemostatic valve side port 106. The catheter system can further include an intracranial balloon 89 disposed within the inner lumen of the inner aspiration catheter shaft, a fourth hemostatic valve 86 connected to the inner aspiration catheter shaft, with the fourth hemostatic valve having a side port, a fifth hemostatic valve 50 connected to the fourth hemostatic valve and the fifth hemostatic valve has a side port. There can be a microwire 87 disposed within the inner lumen and the intracranial balloon. A side gap can also be positioned proximally to the balloon.

The catheter system 10 can also be embodied with a balloon aspiration catheter 100 that includes an inflation means, such as a balloon 107, having a body with a distal tip 144 and a proximal end 142 thereof, and an inflation means lumen within the body, such as secondary lumen 110. There can be a primary aspiration means connected to the balloon lumen 110, such as pump 60. There is an inlet means disposed along the body of the inflation means, such as inlets 103. A secondary inflation means can connected to the body of the inflation means, such as outer balloon aspiration catheter 100, and an inner aspiration catheter having an inner lumen, the inner aspiration catheter configured to slide within the inflation means lumen and selectively past and occluding the inlet means.

The system can include an aspiration means for selectively aspirating the inflation means, such as pump 60, and also include a pumping means for selectively pumping a fluid into and out of the inflation means, such as syringe 59. The balloon is configured such that the balloon inflates when the inner aspiration catheter 100 is advanced beyond the balloon inlets 103 and a solution is injected into a secondary inflation channel, such as catheter 100. Further, the inner aspiration catheter 200 can be compliant and configured to atraumatically advance within the balloon lumen.

The balloon 107 can be configured to automatically deflate with retrieval of the inner aspiration catheter from the balloon primary lumen 101. Further, the system 10 can include a plurality of radiopaque markers 104 disposed throughout all lumens.

As discussed herein, the disclosure relates generally to catheter systems, and more particularly, to a balloon assisted thrombectomy dual aspiration and distal access catheter system for neurovascular thrombectomy and intracranial emboli protection. The inclusion of a distal balloon attached to an aspiration catheter that is used alone or in conjunction with a coaxial inner aspiration catheter allows for more distal direct aspiration augmented by distal flow arrest and suction under more subsequently powerful aspiration. This dual function device simplifies the procedure and serves as a uniform platform for stiffer devices including intracranial stents and angioplasty balloons and that can be used in combination with other stroke thrombectomy devices.

It is understood that similarly numbered and/or named components may function in a substantially similar fashion. Redundant explanation of these components has been omitted for clarity. The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A catheter system, comprising:
a first hemostatic valve having a first hemostatic valve side port and a proximal end;
an inner aspiration catheter hub connected to the proximal end of the first hemostatic valve;
an inner aspiration catheter shaft connected to the inner aspiration catheter hub the inner aspiration catheter shaft having an interior thereof, including:
an inner lumen formed within the interior of the inner aspiration catheter shaft,
an inner lining fitted within the inner lumen, the inner lining having an outer lining disposed over the inner lining, and
a stress relief jacket disposed fitted over the outer lining;
an outer balloon aspiration catheter hub, including:
a second hemostatic valve having a second hemostatic valve side port, and
a third hemostatic valve having a third hemostatic valve side port (106); and
an outer balloon aspiration catheter shaft having an interior thereof, and including:
a primary lumen formed within the interior of the outer balloon aspiration catheter shaft and connected to the second hemostatic valve, and the primary lumen further having an exterior thereof,
a secondary lumen fitted over the primary lumen and connected to the third hemostatic valve, wherein the secondary lumen has a secondary lumen side port and an interior thereof, and
an expandable balloon formed within the interior of the secondary lumen, the balloon having an interior thereof and a plurality of balloon side ports disposed along the interior of the balloon and a portion of the exterior of the primary lumen, wherein the plurality of balloon side ports connect the primary lumen to the secondary lumen.

2. The system of claim 1, wherein the inner aspiration catheter shaft is hydrophilic.

3. The system of claim 1, wherein the inner aspiration catheter shaft has a lubricious outer coating.

4. The system of claim 1, wherein a plurality of radiopaque markers are disposed throughout the inner lumen, the primary lumen, and the secondary lumen.

5. The system of claim 1, wherein the first hemostatic valve, the second hemostatic valve, and the third hemostatic valve each include a luer fitting.

6. The system of claim 1, wherein the first hemostatic valve, the second hemostatic valve, and the third hemostatic valve each include at least one of a one-way stop cock and a three-way stop cock.

7. The system of claim 6, further including a machine pump connected to the at least one of the one-way stop cock and three-way stop cock.

8. The system of claim 6, further including a syringe connected to at least one of the one-way stop cock and three-way stop cock.

9. The system of claim 8, wherein the syringe is an aspiration syringe.

10. The system of claim 1, wherein the inner aspiration catheter hub further comprises at least two hub side wings.

11. A catheter system, comprising:
a balloon aspiration catheter including:
a balloon having a body with a distal tip and a proximal end thereof, and a balloon lumen within the body,
a primary aspiration channel connected to the balloon lumen,
a plurality of balloon inlets disposed along the body of the balloon, and
a secondary inflation channel connected to the body of the balloon; and
an inner aspiration catheter having an inner lumen, the inner aspiration catheter configured to slide within the balloon lumen and selectively past and occluding the balloon inlets.

12. The system of claim 11, wherein the balloon is configured such that the balloon inflates when the inner aspiration catheter is advanced beyond the balloon inlets and a solution is injected into the secondary inflation channel.

13. The system of claim 12, wherein the balloon of the balloon aspiration catheter is further configured to arrest flow through the balloon lumen.

14. The system of claim 12, wherein the inner aspiration catheter is compliant and configured to atraumatically advance within the balloon lumen.

15. The system of claim 11, wherein the balloon is configured to automatically deflate with retrieval of the inner aspiration catheter from the balloon lumen.

16. The system of claim 11, further including a plurality of radiopaque markers disposed throughout the balloon lumen and inner lumen.

17. The system of claim 11, further including a machine pump connected to the balloon lumen.

18. A catheter system, comprising:
a balloon aspiration catheter including:
an inflation means having a body with a distal tip and a proximal end thereof, and an inflation means lumen within the body,
a primary aspiration means connected to the balloon lumen,
an inlet means disposed along the body of the inflation means, and
a secondary inflation means connected to the body of the inflation means; and
an inner aspiration catheter having an inner lumen, the inner aspiration catheter configured to slide within the inflation means lumen and selectively past and occluding the inlet means.

19. The system of claim 18, further including an aspiration means for selectively aspirating the inflation means.

20. The system of claim 18, further including a pumping means for selectively pumping a fluid into and out of the inflation means lumen.
